(19) United States
(12) Patent Application Publication
(10) Pub. No.: US 2006/0233886 A1
(43) Pub. Date: Oct. 19, 2006

(54) Antimicrobial composition and method for making same

(76) Inventors: Andrew Kielbania JR., Chalfont, PA (US); Roland John Crowther, Auckland (NZ)

Correspondence Address: REED SMITH, LLP;ATTN: PATENT RECORDS DEPARTMENT, 599 LEXINGTON AVENUE, 29TH FLOOR, NEW YORK, NY, 10022-7650, US

(21) Appl. No.: 11/381,269

(22) Filed: May 2, 2006

Related U.S. Application Data
(63) Continuation-in-part of application No. 11/095,329, filed on Mar. 31, 2005.

(51) Int. Cl. A61K 33/42; A61K 33/40; A61K 31/19

(52) U.S. Cl. 424/601; 424/616; 514/557

(57) ABSTRACT

Anti-microbial Formulations and methods of their use and production are disclosed. The Formulations of the present invention are effective as broad spectrum anti-bacterial agents with efficacy against both Gram-negative and Gram-positive bacteria, as anti-viral agents with efficacy against both enveloped and non-enveloped viruses, anti fungal agents and anti spore forming agents. The present invention includes anti-microbial Formulations that include at least one surfactant, optionally at least one acid, at least one non-cationic anti-microbial agent, and optionally water. The anti-microbial Formulations of the present invention may additionally contain an organic salt. The organic salt may be a salt of the same acid that is used in the Formulation or a salt of a different acid.

### CROSS REFERENCE TO RELATED APPLICATIONS

This application is a continuation-in-part of application Ser. No. 11/095,329, filed Mar. 31, 2005, the entire contents of which are incorporated herein by reference.

### BACKGROUND OF THE INVENTION

1. Field of the Invention

The present invention relates to chemical Formulations that possess anti-bacterial, antiviral and anti-fungal properties and methods of making and using those Formulations.

2. Description of the Background

Bacteria, viruses and fungi, e.g., mold, are a major source of disease and contamination throughout modern society. The need to control the growth of these micro-organisms is paramount for maintaining public health as well as reducing costly commercial and industrial contamination. In the case of bacteria, infections and contaminations are effected by both Gram-positive (e.g., Staphylococcus aureus) and Gram-negative (e.g., Escherichia coli) bacteria. Many anti-bacterial agents are limited in their efficacy to only one of those two classes of bacteria. Moreover, known anti-viral agents are often effective only against enveloped or non-enveloped viruses, but not both.

While numerous anti-microbial agents exist, many have the further limitation that they cannot be compounded into Formulations for extended transport over concerns regarding degradation of stability and/or efficacy. Thus, there has been a long standing need for stable and efficacious anti-microbial Formulations that possess broad spectrum activity.

### Definitions

As used herein, unless otherwise indicated, the following terms mean:

Microbe(s) includes bacteria, viruses, fungi, mold, and spore-forming bacteria;

Inhibit means substantially stopping the growth of a Microbe(s);

Kill means substantially causing the death of a Microbe(s);

Anti-microbial agent(s) means a compound or composition which in liquid, solid or gaseous form, Inhibits or Kills Microbes.

### SUMMARY OF THE INVENTION

The present invention generally relates to anti-microbial Formulations and methods of their use and production. The Formulations of the present invention are effective as broad spectrum anti-bacterial agents with efficacy against both Gram-negative and Gram-positive bacteria, as anti-viral agents with efficacy against both enveloped and non-enveloped viruses, anti fungal agents and anti spore forming agents. The present invention includes anti-microbial Formulations that include at least one surfactant, optionally at least one acid, at least one non-cationic anti-microbial agent, and optionally water. The anti-microbial Formulations of the present invention may additionally contain an organic salt. The organic salt may be a salt of the same acid that is used in the Formulation or a salt of a different acid. More particularly, we have discovered an anti-microbial composition comprising:
at least one surfactant present in a concentration from about 3% to about 95% by weight;
an acid present in a concentration of up to about 20% by weight;
at least one non-cationic anti-microbial agent and
the balance water,
wherein the amount of anti-microbial agent is effective to produce a Zone of Inhibition of from 15 to 150 mm and for the composition to exhibit Plate Cidality against each of Staphylococcus aureus ATCC 6538, Escherichia coli ATCC 8739, Pseudomonas aeruginosa ATCC 9027 and Candida albicans ATCC 10231 and exhibits a negative cytopathic effect (is CPE negative) at dilutions up to 1:2000.

In one embodiment, we have discovered that the inventive efficacious antimicrobial Formulations can be prepared when a solid antimicrobial agent is introduced into the Formulation above its melting point and a minimum level of surfactant is present in the Formulation to provide a clear and homogeneous Formulation both initially and upon sequential dilution with water. A straight forward and easy to use procedure is described to determine the required minimum level of surfactant and to determine whether the Formulation has the necessary features and characteristics required for an efficacious antimicrobial Formulation.

In yet another embodiment, a solid antimicrobial is combined with another liquid antimicrobial agent that can dissolve or liquefy the solid antimicrobial agent. In this embodiment, it is not necessary to employ heat to melt the solid antimicrobial agent. However, in this embodiment, a clear, homogeneous Formulation is obtained which on sequential dilution exhibits a high degree of antimicrobial efficacy. In particular, if a peroxide is used as a part of the antimicrobial agent, then it is possible to dissolve the solid antimicrobial agent in water containing the peroxide at a temperature below its melting point and still achieve the characteristic antimicrobial properties of the inventive composition.

### DETAILED DESCRIPTION OF THE INVENTION

The present invention broadly relates to anti-microbial compositions comprising at least one surfactant, optionally at least one acid, at least one non-cationic anti-microbial agent, and optionally water. The anti-microbial Formulations of the present invention may additionally contain an organic salt. The organic salt may be a salt of the same acid that is used in the Formulation or a salt of a different acid.

The Formulations of the present invention includes a surfactant. The surfactant used in the present Formulation may be amphoteric, cationic, nonionic or anionic. The most preferred surfactants are amphoteric members of the following classes of chemical compounds: alkylamine oxides, alkyamidopropyl amine oxides, alkyl betaines, alkyamidopropyl betaines, and sultaines. Specific examples of alkyl amine oxides that may be used in the present invention include octyl amine oxide, decyl amine oxide, lauryl amine oxide, iso-dodecyl amine oxide, myristyl amine oxide, cetyl amine oxide, oleamine oxide, stearyl amine oxide, and palmitamine oxide. Specific examples of alkylamidopropyl amine oxides that may be used in the present invention include laurylamidopropyl amine oxide, cocamidopropyl amine oxide, stearamidopropyl amine oxide, germamidopropyl amine oxide. Specific examples of alkyl betaines that may be used in the present invention include octyl betaine, lauryl betaine, cocobetaine, cetyl betaine, oleyl betaine, and tallow dihydroxylethyl glycinate. Specific examples of alkylamidopropyl betaines that may be used in the present invention include caprylamidopropyl betaine, capramidopropyl betaine, lauamidopropyl betaine, cocamidopropyl betaine, isostearamidopropyl betaine, wheatgermamidopropyl betaine, and coco/sunfloweramidopropyl betaine. Specific examples of sultaines that may be used in the present invention include cocamidopropyl hydroxysultaine and lauryl hydroxysultaine. The examples provided above are not an exhaustive list of the surfactants that may be used in the present invention. One of skill in the art will recognize additional members and variations within the various categories listed above. Such additional compounds are considered to be within the scope of the present invention.

A single surfactant of the types listed above may be used in the Formulations. Alternatively, Formulations that include multiple surfactants are also considered as within the scope of the present invention.

Particularly preferred surfactants that may be used in the present invention include alkali metal salts of alkylamphoacetates, dialkali metal salts of alkylamphoacetates, alkali metal salts of alkylamphopropionates, dialkali metal salts of alkylamphopropionates, alkanolamides, disodium alkyl sulfosuccinates, monsodium diakyl sulfosuccinates, disodium alkylethoxy sulfosuccinates, sodium ethoxydimethicone sulfosuccinates, disodium propoxydimethicone sulfosuccinates, disodium alkylamido MEA sulfosuccinates, disodium alkylamido MPA sulfosuccinates, sodium alkylamidopropoxy sulfosuccinates, alkylamidopropylmorpholine lactate, and ethylene oxide/propylene oxide diblock and triblock surfactants.

The antimicrobial agents that are preferably employed in the present invention are non-cationic antimicrobial agents. The non-cationic antimicrobial agents may be selected from the groups of phenolics, halogenated phenolics, halogenated diphenyl ethers, halogenated carbonilides, water soluble- or water insoluble peroxy oxidizing agents (e.g. peroxides, peresters, peracids, persulfates), or mixtures thereof.

More specifically, typical antimicrobials in this include:

Phenolics:
Phenol—melting point 43° C.
Xylenol—melting point 75° C.
2-nitrophenol—melting point 45° C.
2-phenyl phenol—melting point 57-59° C.

Halogenated Phenolics:
2,3-dichlorophenol—melting point 55-57° C.
2,4-dichlorophenol—melting point 42-43° C.

Halogenated Diphenyl Ethers
Triclosan—melting point 56-58° C.

Peroxides: 4
Dicumyl peroxide—melting point 39-41° C.
Lauroyl peroxide—melting point 53-57° C.
t-butylhydroperoxide and hydrogen peroxide

Peracids:
Peracetic acid—melting point 56° C.

Peresters:
t-butyl peracetate—melting point 38° C.
t-butyl perbenzoate—melting point 93° C.

Persulfates:
Sodium persulfate—melting point 100° C.

The Formulations of the present invention may also include a wetting agent at a concentration of up to about 3%, by weight. For example, commercially available wetting agents, such as, Fluorocarbon and silicone based wetting agents are particularly effective.

The Formulations of the present invention containing the various components indicated hereinabove may be in the form of a liquid, viscous liquid, liquid, e.g., a liquid soap, a pasty mixture, e.g., a heavy-duty soap used by mechanics, or a semi-solid or solid, e.g., a bar of soap. This depends on the solids content of the Formulation and the present invention contemplates all of such forms. However, the form of the inventive composition does not affect its antimicrobial properties. The inventive formulation, in such forms, may have components in the following amounts (All percent weights herein are based on the total weight of the Formulation):

| | | | Preferred | | Most Preferred | |
|---|---|---|---|---|---|---|
| Formulation | Range | | Range | | Range | |
| Component | Liquid¹ | Solid² | Liquid | Solid | Liquid | Solid |
| (% by weight) | Form | Form | Form | Form | Form | Form |
| Solids % | 1-50 | 50-98 | 10-50 | 60-98 | 15-50 | 50-95 |
| by weight | | | | | | |

| | | | |
|---|---|---|---|
| Surfactant | 0.2-98 | 0.4-50 | 5-30 |
| First Solid | 0.0-25 | 0.5-18 | 0.1-8.0 |
| Antimicrobial | | | |
| Agent | | | |
| Second Liquid | 0-30 | 0-20 | 0-15 |
| Antimicrobial | | | |
| Agent | | | |
| Acid | 0-20 | 0-12 | 1-10 |
| Acid salt | 0-7 | 0-5 | 0-3 |
| ppm Transition | 0.0-a catalytica [symbol] | 5-500 | 10-500 |
| Metal Ion | effective amount | | |

¹Solid at room temperature
²Liquid at room temperature
³Only needed if a peroxy antimicrobial agent is present. Includes metal ions such as iron, copper, manganese, chromium, cobalt, nickel, vanadium, tungsten, molybdenum and combinations thereof.

Occasionally peroxy compounds such as hydrogen peroxide, t-butyl hydroperoxide, benzoyl peroxide and peracetic acid are inactive or exhibit retarded reactions in the absence of catalytic amounts of certain transition metal ions. For example, in radical emulsion polymerization of unsaturated acrylic monomers using hydrogen peroxide or t-butyl hydroperoxide a small amount of aqueous iron sulfate solution is added to insure the peroxy compounds are active and polymerization initiates as soon as the peroxy compound is added to the reactor. In the preparation of our antimicrobial formulations, a catalytically effective amount of a transition metal ion, such as, iron sulfate may be included to achieve the desired antimicrobial effect. Besides iron, copper, manganese, chromium, cobalt, nickel, vanadium, tungsten or molybdenum ions or combinations may be used.

Peroxy compounds such as hydrogen peroxide or peracetic acid are especially useful to further enhance the efficacy of antimicrobial formulations against difficult to control microorganisms, especially spore forming microorganisms. However, these types of peroxy compounds may exhibit instability in the presence of antagonistic compounds, such as surfactants, which have chemical functionality that can react with the peroxy compound. For example, surfactants based upon natural feedstocks like coconut and palm oils, such as cocobetaines, contain carbon double bonds and conjugated unsaturation which peracetic acid will react with. Often this reactivity or instability of peracetic acid is evident by degassing of the formulation and pressure build up in storage containers. This instability of peracetic acid in the presence of other functional organic materials may require the peracetic acid formulation be used immediately after preparation because of its short active life span or must be mixed at point of use. The mixing at point of use presents several handling and safety issues in view of peracetic acid's oxidizing ability, corrosiveness and toxicity. Therefore there is a need to develop a very potent and stable peroxy containing antimicrobial formulation that is easy to apply and use. We have developed a process which results in highly efficacious and stable peroxy antimicrobial formulations. These new formulations can contain mixtures of non cationic and nonperoxy antimicrobial agents, such as Triclosan, with one or more peroxy antimicrobial agents such as hydrogen peroxide, t-butyl hydroperoxide, peracetic acid, and the like.

The stable peroxy formulation are prepared by initially adding from about 15 to 50% by weight of stabilized hydrogen peroxide solution to a surfactant, an optional acid, and water. The mixture is then heated at approximately 60° C. for about 8 to 12 minutes. Thereafter, a noncationic antimicrobial agent is added with rapid stirring. In about 1-4 minutes a clear homogenous low viscosity solution forms. The solution is stirred at about 60-63° C. for 10 minutes. The formulation is cooled to room temperature. The peroxide component, for example, hydrogen peroxide, t-butyl hydroperoxide, peracetic acid, and the like may then be added and the mixture is stirred from one to five minutes to form a clear and homogeneous solution which will not degas. Further dilution of the formulation with stabilized hydrogen peroxide solution or an aqueous solution of iron sulfate provides additional stability or efficacy. The formulation can also be diluted with an aqueous solution of iron sulfate or water.

The Formulations of the present invention may include acid at a concentration of up to about 20%, by weight. The acid may be an organic acid such as citric acid or an inorganic acid such as phosphoric acid, a mono or multi salt of the acids, e.g., sodium, potassium, ammonium, lithium, and the like. Such acids and acid salts function as a buffer for the Formulation. The balance of the Formulations of the present invention is preferably water in a percentage by weight that is determined by the percentages of the various components that are specified above. The percent solids or percent nonvolatiles components in the Formulation may be up to 98%.

The Formulations of the present invention may be prepared by two different methods depending on the form of the antimicrobial agent, i.e., whether it is a solid or liquid at ambient temperatures. In a first preparative method wherein the antimicrobial agent is solid, a container may be charged with water, surfactant, and optionally, an organic acid for pH control. The contents of the container are stirred and heated to the temperature of the melting point of the solid antimicrobial agent to be used. A wetting agent may be added at that step in the process. The solid non-cationic anti-microbial agent is added to the heated mixture with rapid stirring. The solid anti-microbial agent, should be allowed to dissolve fully. After the anti-microbial agent is fully dissolved, the solution is stirred at this temperature for about 20 minutes.

In an alternative embodiment, if the surfactant is also a solid, the solid antimicrobial agent and solid surfactant are added to the container without water or acid and the container heated to convert the a antimicrobial agent to a liquid which then dissolves the surfactant. The mixture is then stirred to fully intermix the components. Water can then be added along with acid as required for pH adjustment, and the mixture cooled to ambient temperature. The pH is in a range from about 2.2 to 8.3.

In the second preparative method, a first solid antimicrobial agent which is a solid at ambient temperature is admixed with a second antimicrobial agent which is a liquid at ambient temperature to dissolve the solid antimicrobial in the liquid agent. Alternatively, if the liquid antimicrobial agent does not fully dissolve the solid agent, water or a mixture of water and surfactant may be added to complete the dissolution.

The antimicrobial agent may be formulated as high solids products, e.g. 95% or more solids for example in the form of a soap bar, which when moistened with water exert the antimicrobial effect. Alternatively, it can be formulated in aqueous dilutions of varying proportions depending on the particular end use contemplated.

An important property of the present invention is that the Formulations produced as described above when mixed with water produce homogeneous, clear solutions, and display superior stability making them particularly useful for a wide variety of antimicrobial applications. Inasmuch as the microbe to be inhibited or killed are present in aqueous phase, the homogeneity of the resulting solutions in critical to producing the high antimicrobial efficacy of the present invention. The resulting diluted or full strength Formulations may be tested for anti-microbial activity in the following manner.

The anti-microbial activity of the Formulations of the present invention is expressed herein in three ways:

1. Zone of Inhibition

The Zone of Inhibition of the inventive formulation is determined as follows:

disks impregnated with known concentrations of antimicrobial Formulations are placed onto the surface of Mueller-Hinton Agar 150 millimeter plates that have been freshly seeded with a known quantity of bacteria (varying from 10/5 to 10/8 CFU/ml). a variety of bacteria can be challenged in this manner in order to determine the spectrum of activity of the antimicrobial agent. The standard inoculum is a 0.5 McFarland standard turbidity, which is approximately 1.5×10/8 CFU/ml. The surface of the agar is swabbed in three directions to ensure an even and complete distribution of inoculum over the entire plate. Within 15 minutes of inoculation, the antimicrobial agent disks are applied and the plates are inverted for incubation to avoid accumulation of moisture on the agar surface that might interfere with the interpretation of the test results. For most organisms, incubation is at 35° C. in air but increased CO₂ is used with certain fastidious bacteria. The dynamics and timing of antimicrobial agent diffusion to establish a concentration gradient coupled with the growth of organisms over an 18- to 24-hour duration is desired to obtain reliable results. Therefore, incubation of disks beyond that allotted time was preferably avoided.

Using a dark background and a reflected light, the plate is situated so that a ruler or caliper may be used to measure inhibition zone diameters for each antimicrobial agent. Zones are recorded in millimeters (mm) of diameter. Known controls (including media controls) are employed with each run to assure accuracy of techniques employed.

2. Plate Cidality

A loop full of inoculum is taken within the Zone of Inhibition as measured above approximately 2 millimeters from the disk edge after the plates have been incubated for 18-24 hours. The loop is inoculated to a fresh Trypticase Soy Agar plate and then incubated at 35° C. for 18-24 hours before being read. The results were recorded as either “No growth”, indicating Plate Cidality or “Growth” indicating no Plate Cidality. Alternatively, the number of colonies was recorded in cases where there was incomplete Plate Cidality.

3. Antiviral Activity

The antiviral activity of the inventive formulation is expressed as a negative or positive CPE. CPE negative means that the Formulation allows no growth of an ATCC culture of Herpes Simplex virus: CPE positive means that the formulation is not effective and allows growth under the test conditions. The test to determine whether the formulation is CPE positive or negative is carried out as follows:

MRC-5 human embryonic diploid lung fibroblast cells are inoculated with a previously germicide treated ATCC culture of Herpes Simplex virus maintained in Eagle's minimum essential medium. The treatment is with the Formulation to be tested at a 1:3 dilution held at room temperature for 1 hour prior to cell inoculation. Two shell vials are inoculated in this manner and then incubated at 35 C, one for 24 hours and the other for 48 hours. After incubation they are observed for cytopathic effect (CPE) using standard immunofluorescent staining. CPE negative is considered to be an effective kill of HSV. CPE positive is considered to be a failure. Positive and negative controls are run with all experiments.

As noted above, an important property of the inventive formulation is that when diluted with water it provides a clear homogeneous mixture which, in turn, allows it to be an effective and efficient anti-microbial agent. Table 1 sets forth the ratios and amounts of surfactant and solid antimicrobial agent to achieve a clear homogeneous mixture on sequential aqueous dilution using the first embodiment outlined hereinbefore wherein the surfactant is cocobetaine (low salt version) or a mixture of cocobetaine (low salt version) and lauryl amine oxide and the solid antimicrobial is triclosan (triclosan is 2,4,4′-trichloro-2′-hydroxydiphenyl ether; it is also known as 5-chloro-2-(2,4-dichlorophenoxy)phenol).

TABLE 1
Requirements for Clear Homogeneous
Antimicrobial Formulation Both Initially and Upon Sequential Dilution

| | | Surfactants/ | | | |
|---|---|---|---|---|---|
| | | Triclosan | | | |
| Formulation | Surfactants | Ratio⁶ | Temperature | Initial | Sequential |
| No.⁴ | weight %⁵ | (By Wt.) | ° C.⁷ | Clarity | Dilution Clarity |
| 22 | 24% CB-LS/2% LAO | 6.9 | 60 | YES | YES |
| 5 | 25.7% CB- | 5.9 | 60 | YES | YES |
| | LS/0.9% LAO | | | | |
| 1 | 12% CB- | 5.6 | 60 | YES | YES |
| | LS/0.4% LAO | | | | |
| 2 | 12% CB- | 5.6 | 60 | YES | YES |
| | LS/0.4% LAO | | | | |
| 45 | 12% CB- | 5.6 | RT | NO | — |
| | LS/0.4% LAO | | | | |
| 23 | 12% CB-LS | 5.6 | 60 | YES | YES |
| 24 | 25% CB- | 5.2 | 60 | YES | YES |
| | LS/1.3% LAO | | | | |
| 25 | 8.8% CB- | 4.1 | 60 | YES | YES/borderline |
| | LS/0.3% LAO | | | | |
| 26 | 7.7% CB- | 3.6 | 60 | NO | |
| | LS/0.3% LAO | | | | |
| 27 | 6.6% CB- | 3.1 | 60 | NO | |
| | LS/0.2% LAO | | | | |
| 28 | 9% CB | 1.2 | 60 | NO | |
| 29 | 12% CAB/0.4% LAO | 5.6 | 60 | YES | YES |
| 30 | 12% CAB | 5.5 | 60 | YES | YES |
| 31 | 9.9% CAB | 4.5 | 60 | BL | Borderline |
| 32 | 8.8% CAB | 4.0 | 60 | NO | — |
| 33 | 7.7% CAB | 3.5 | 60 | NO | — |
| 6 | 43.5% OB/2.2% LAO | 11.9 | 60 | YES | Borderline/clear⁸ |
| 34 | 35.4% OB/7.1% LAO | 11.3 | 60 | YES | YES/borderline |
| 35 | 22.5% OB/4.5% LAO | 9.0 | 60 | YES | NO |
| 36 | 25% OB/2.5% LAO | 7.6 | 60 | YES | NO |
| 37 | 9% OB | 1.2 | 60 | NO | — |
| 38 | 17.7% LAO | 8.0 | 60 | YES | YES |
| 39 | 9% LAO | 6.0 | 60 | NO | — |
| 46 | 9% LAO | 6.0 | RT | NO | — |
| 40 | 21% LAO | 1.2 | 60 | NO | — |
| 41 | 24% LB/0.8% LAO | 6.5 | 60 | YES | YES |
| 42 | 12.5% LB/1.3% LAO | 3.6 | 60 | NO | — |
| 43 | 37.7% OAO | 11.1 | 60 | NO | — |
| 44 | 4.5% MAO/4.5% CAB | 6.0 | RT | NO | — |

⁴Formulation numbers refer to the Formulations of the examples.
⁵Abbreviations of surfactants: CB-LS = cocobetaine, low salt version; LAO = lauryl amine oxide; CAB = cocamidopropyl betaine; OB = octyl betaine; LB = lauryl betaine; OAO = octyl amine oxide; MAO = myristyl amine oxide
⁶The weight ratio of total surfactants to triclosan.
⁷60° C. means 58-63° C.; RT means room temperature.
⁸Initially the Formulation was borderline upon sequential dilution but became a clear homogeneous solution upon standing at room temperature.

Table 1 shows that not only is a certain minimum ratio of total surfactant(s) to Triclosan necessary to obtain a clear homogeneous Formulation solution but also the antimicrobial agent—in this case Triclosan—must be introduced into the Formulation above its melting point, e.g., 57-58° C. for Triclosan. The clarity characteristics of the Formulations are shown in the last two columns of Table 1. It is also evident that some surfactants or surfactant combinations are more effective at giving clear homogeneous Formulations than other surfactants. It is well understood by those skilled in the art that all surfactants are not equally effective. For example, the cocobetaine—low salt version the weight ratio of the surfactant to Triclosan of about 4.1 is sufficient to give a clear homogeneous Formulation while for cocamidopropyl betaine, octyl amine oxide and lauryl oxide, higher levels of surfactant are required.

The last two columns of Table I sets forth data indicating how effectively Triclosan has been incorporated into the Formulation, how effectively the Formulation will transport Triclosan to take advantage of its antimicrobial activity, and potential antimicrobial efficacy of the Formulation. This analysis as detailed hereinbelow involves a visual inspection and therefore does not need any special or expensive equipment.

A. Analysis of the Initial Clarity:

After preparation, the Formulation is visually examined at room temperature to determine if it is a clear and homogeneous solution. A “Yes” classification is assigned if the Formulation is a clear and homogenous solution with no signs of solid particulates, oily material or any type of second phase. A “No” classification is assigned if the formulation is not clear, homogeneous, is a blue or white dispersion or emulsion or has a solid precipitate, or has an oily phase separated from the aqueous phase.

B. Analysis of the Clarity of the Sequentially Diluted Formulation

This involves visual examination of the formulation after sequential dilutions with water at ambient temperature. The analysis is carried out as follows:

To 1 gram of the Formulation being evaluated in a clear container, 4 grams of water are added to give a 1:5 dilution. The contents of the container are mixed manually for about one minute. The contents are visually inspected and assigned one of the following rankings: yes; yes/borderline; no/borderline; no. If a no or a no/borderline ranking is assigned, the analysis is complete and no further dilution steps are necessary. If a yes or yes/borderline ranking is assigned additional sequential dilutions by adding water to achieve the following dilutions:

5 g water—1:10 dilution

5 g water—1:15 dilution

5 g water—1:20 dilution

10 g water—1:30 dilution

20 g water—1:50 dilution

The ranking assigned is given based on the following criteria:

Yes—the solution is clear and homogenous, no visual detection of solid or oil phase evident.

Yes/borderline—the solution has a very slight blue tint but has no signs of gross solid particles or oil phase.

No/borderline—the solution has a blue to slightly white appearance, the solution is no longer clear.

No—a white dispersion, emulsion or suspension is present, or solid has precipitated out, or a second liquid/oil phase is present.

The no or no/borderline ranking indicates that the Formulation is not sufficiently robust to deliver/transport the Triclosan and the Formulation will not be an effective antimicrobial. The Formulations that have been assigned a yes or yes/borderline ranking have a level of surfactant present which is sufficient to both keep the Triclosan in aqueous solution (prevent phase separation) and to deliver/transport the Triclosan effectively and give an efficacious antimicrobial as shown in Table 2. In those cases where sufficient surfactant is present, there is no need to incorporate solvents to dissolve the Triclosan into the Formulation. In addition there is no need to incorporate hydrotropes to aid in the delivery/transport of the Triclosan in the antimicrobial Formulation. It is highly desirable to eliminate solvents from Triclosan antimicrobial Formulation because to their toxicity, VOC (volatile organic compound) pollution and fire hazard issues, and irritation characteristics. Many of these solvents are harsh to human skin and eyes and are detriments to antimicrobial Formulations. In addition the present of solvents poses hazards and limits the useful utility in large scale, household, commercial, institutional and industrial settings. In addition it is highly desirable to eliminate hydrotropes from antimicrobial Formulations. Hydrotropes are very strong acids or salts that are irritants, corrosive and harsh to human skin and eyes. The present invention eliminates the need to use solvents and/or hydrotropes in antimicrobial Formulation by incorporating the Triclosan into the Formulation above the melting point of Triclosan or by using a second liquid antimicrobial agent to dissolve/liquefy the Triclosan and by using a sufficient amount of surfactant to both incorporate the Triclosan into the Formulation and to keep the Triclosan in the Formulation upon dilution. These features are indicative of effective delivery/transport of the Triclosan to give an efficacious antimicrobial. The surfactants used in the present invention are preferably very mild and can mitigate irritation to human skin and eyes.

TABLE 2
Antimicrobial Efficacy for Clear and
Homogeneous Formulations

| | | |
|---|---|---|
| | Zone of | |
| Formulation | Inhibition (mm) | Plate Cidality |

| No. | S. aureus | E. coli | S. aureus | E. coli |
|---|---|---|---|---|
| Formulation 5 | 52 | 50 | YES | YES |
| Formulation 1 | 51 | 42 | YES | YES |
| Formulation 2 | 53 | 42 | YES | YES |
| Formulation 6 | 45 | 37 | YES | YES |

S. aureus = Staphylococcus aureus,
E. coli = Escherichia coli

Table 2 shows that antimicrobial Formulations which are initially clear and homogeneous and remain clear and homogeneous upon sequential dilution have a high degree of antimicrobial efficacy. While the prior art has employed high levels of hydrotropes or combinations of solvent and hydrotropes, we have achieved exceptional antimicrobial efficacy with a combination of the right level of surfactant and the right processing temperature in a easy straight forward process. Elimination of solvents and hydrotropes is significant since these materials can be flammable, toxic, corrosive or irritants to the skin and eyes thereby limiting broad industrial, commercial, institutional and household use.

TABLE 3
Inadequate Antimicrobial Efficacy for
Formulations Which Are Not Clear and Homogeneous

| | | |
|---|---|---|
| | Zone of | |
| Formulation | Inhibition (mm) | Plate Cidality |

| No. | S. aureus | E. coli | S. aureus | E. coli |
|---|---|---|---|---|
| 34 | 45 | 37 | NO | YES |
| 35 | 42 | 33 | NO | YES |
| 39 | 49 | 35 | NO | YES |
| 46 | 42 | 36 | NO | YES |
| 43 | 38 | 30 | NO | YES |
| 44 | 49 | 43 | NO | NO |

S. aureus = Staphylococcus aureus,
E. coli = Escherichia coli

Table 3 shows that antimicrobial Formulations which are not initially clear and homogeneous or do not remain clear and homogeneous upon sequential dilution have inadequate antimicrobial efficacy. These Formulations have smaller zones of inhibition and fail to kill Staphylococcus aureus or fail to kill both Staphylococcus aureus and Escherichia coli. These antimicrobial Formulations lack broad efficacy. Those skilled in the art can determine the minimum required surfactant level by using the techniques described above to determine both the initial Formulation clarity and homogeneity and the clarity and homogeneity of sequentially diluted material on a series of Formulations where the surfactant or surfactant combination has been incrementally increased over a series of Formulations. The minimum level of surfactant necessary is determined at the point where both the Formulation and sequentially diluted Formulations are both clear and homogeneous.

In addition, the antimicrobial Formulations of the present invention are effective at high dilution. Table 4 shows the Minimum Inhibitory Concentration (MIC) and the Minimum Bacteriacidal Concentration (MBC) values determined for two of the Formulations of the present invention against four microbes and compared to a commercial antibacterial hand soap containing Triclosan. The MIC values were determined using a standard broth method with sequential two fold dilutions. The MIC's were determined by visual inspection at the point of no turbidity.

TABLE 4
Minimum Inhibitory Concentrations

| Antimicrobial | Organism | MIC/MBC |
|---|---|---|
| Dial ® | Staphylococcus | <1:10/static |
| Antibacterial | epidermidis | |
| Soap | | |
| | Corynebacterium | 1:10/static |
| | jeikeium | |
| | Micrococcus luteus | <1:10/static |
| | Propionibacterium | <1:10/static |
| | acnes | |
| Formulation 10 | Staphylococcus | >1:1280/>1:1280 |
| | epidermidis | |
| | Corynebacterium | >1:1280/>1:1280 |
| | jeikeium | |
| | Micrococcus luteus | 1:640/1:320 |
| | Propionibacterium | 1:640/1:320 |
| | acnes | |
| Formulation 14 | Staphylococcus | >1:1280/>1:1280 |
| | epidermidis | |
| | Corynebacterium | >1:1280/>1:1280 |
| | jeikeium | |
| | Micrococcus | >1:1280/>1:1280 |
| | luteus | |
| | Propionibacterium | >1:1280/>1:1280 |
| | acnes | |

The first entry above in Table 4 is a commercial antibacterial soap containing Triclosan. It was used as purchased with the first dilution being 1:10. The results preceded by <means the dilution was too great and the MIC was passed. The results preceded by> means the test was stopped but further dilution of the Formulation is still possible. As you can see the efficacy of the two Formulations of the present invention is far superior to the Triclosan commercial comparison. In addition our two Formulations were also cidal to the microorganisms at high dilution (MBC).

The antimicrobial Formulations of the present invention can be used in the concentrated form as prepared or diluted until the minimum concentration which still maintains efficacy is reached. Table 4 shows that dilutions greater than 1:1000 are achievable in the present invention.

Table 5 shows the superior antimicrobial efficacy of Formulations of the present invention compared to a commercial kitchen cleaner, commercial alcohol gel, commercial handsoap with Triclosan, chlorhexidine and betadine solutions.

TABLE 5
Antimicrobial Zone of Inhibition

Zones of Inhibition (mm)

| | Staphylococcus | Pseudomonas |
|---|---|---|
| Antimicrobial Agent | aureus | aeruginsoa |
| Lysol ® kitchen | 24 | CI |
| Cleaner | | |
| Alcohol Gel | Not tested | CI |
| Commercial | Not tested | CI |
| Handsoap with | | |
| Triclosan | | |
| 4% chlorohexidine | Not tested | 16 |
| 10% Betadine | Not tested | 14 |
| Formulation 1 | 51 | 20 |
| Formulation 2 | 53 | 14 |
| Formulation 3 | 41 | 16 |
| Formulation 4 | 50 | 14 |
| Formulation 5 | 52 | 18 |
| Formulation 6 | 45 | 14 |
| Formulation 7 | 57 | 43 |
| Formulation 8 | 112 | 72 |
| Formulation 9 | 40 | 16 |
| Formulation 10 | 61 | 29 |
| Formulation 11 | 61 | 29 |
| Formulation 12 | 24 | Not tested |
| Formulation 13 | 61 | 31 |
| Formulation 14 | 110 | 70 |
| Formulation 15 | 100 | 48 |
| Formulation 16 | 102 | 52 |
| Formulation 17 | 60 | 41 |
| Formulation 18 | 110 | 70 |
| Formulation 19 | 98 | 55 |

CI = contact inhibition only, no Zone of Inhibition detected.

The results in Table 5 show the superior efficacy of the antimicrobial Formulations of the present invention

The following working examples describe presently-preferred embodiments of the present invention.

In the examples, the antimicrobial testing was carried out as follows:

Determination of Zone of Inhibition

Disks impregnated with known concentrations of antimicrobials are placed onto the surface of Mueller-Hinton Agar 150 mm plates that have been freshly seeded with a known quantity of bacteria (varying from 10/5 to 10/8 cfus/ml). Numerous different bacteria can be challenged in this manner in order to determine the spectrum of activity of the agent. The standard inoculum is a 0.5 Mc Farland standard turbidity, which is approximately 1.5×10/8 cfu/ml. The surface of the agar is swabbed in three directions to ensure an even and complete distribution of inoculum over the entire plate. Within 15 minutes of inoculation the antimicrobial agent disk disks are applied and the plates are inverted for incubation to avoid accumulation of moisture on the agar surface that might interfere with the interpretation of the test results. For most organisms, incubation is at 35 C in air but increased CO₂ is used with certain fastidious bacteria. The dynamics and timing of antimicrobial agent diffusion to establish a concentration gradient coupled with the growth of organisms over an 18- to 24-hour duration is critical for reliable results. Therefore incubation of disks beyond that allotted time should be avoided.

Using a dark background and a reflected light the plate is situated so that a ruler or caliper may be used to measure inhibition zone diameters for each antimicrobial agent.

Zones are recorded in millimeters (mm) of diameter. Known controls (including media controls) are employed with each run to assure accuracy of techniques employed. This Zone of Inhibition test was conducted using the formulations in the examples diluted 1 to 3 with water.

Plate Cidality

In order to determine whether the antimicrobial agent being tested had Plate Cidality vs no Plate Cidality, a loop full of inoculum was taken within the Zone of Inhibition approximately 2 mm from the disk edge after the plates have been incubated for 18-24 hours. The loop was inoculated to a fresh Trypticase Soy Agar plate and then incubated at 35 C for 18-24 hours before being read. The results were recorded as “No growth”, indicating Plate Cidality or “Growth” indicating only no Plate Cidality. Alternatively the number of colonies was recorded in cases where there was incomplete Plate Cidality.

Testing for Yeast and Mold Fungi

Disks impregnated with known concentrations of antimicrobials are placed onto the surface of Sabouraud Dextrose Agar 150 mm plates that have been freshly seeded with a known quantity of fungi. Numerous different yeast and mold fungi can be challenged in this manner in order to determine the spectrum of activity of the agent. The standard inoculum is a 0.5 Mc Farland standard turbidity, which is approximately 1.5×10/8 cfu/ml. This concentration is used for yeast fungi. For mold fungi spores are harvested in PBS and using a hemocytometer an approximate count is made guaranteeing an inocula of at least 1.5×10/4 to 10/5 cfu/ml. The surface of the agar is swabbed in three directions to ensure an even and complete distribution of inoculum over the entire plate. Within 15 minutes of inoculation, the antimicrobial agent disk disks are applied and the plates are inverted for incubation to avoid accumulation of moisture on the agar surface that might interfere with the interpretation of the test results. For most organisms, incubation is at 30-35 C in ambient air dependent upon the fungal species. The dynamics and timing of antimicrobial agent diffusion to establish a concentration gradient coupled with the growth of organisms over a 24-48 hours duration for yeast fungi and up to 10 days for mold fungi is critical for reliable results.

Using a dark background and a reflected light the plate is situated so that a ruler or caliper may be used to measure inhibition zone diameters for each antimicrobial agent.

Zones are recorded in millimeters (mm) of diameter. Known controls (including media controls) are employed with each run to assure accuracy of techniques employed.

Plate Cidality for Fungi

In order to determine whether the antimicrobial agent being tested had Plate Cidality vs static, a loopful of inoculum was taken within the Zone of Inhibition approximately 2-4 mm from the disk edge after the plates have been incubated for the designated time. The loop is inoculated to a Sabauroud Dextrose Agar plate and then incubated at 30-5 C dependent upon the fungus for the appropriate time dependent upon whether it is a mold or yeast fungus before being read. The results are recorded as “No growth”, indicating Plate Cidality or “Growth” indicating only no Plate Cidality. Alternatively the number of colonies are to be recorded in cases where there was incomplete Plate Cidality.

Shell Vial Culture for Viral Analysis

MRC-5 human embryonic diploid lung fibroblast cells are inoculated with a previously germicide treated ATCC culture of Herpes Simplex virus maintained in Eagle's minimum essential medium. The treatment is with a germicide to be tested at a 1:3 dilution held at room temperature for 1 hour prior to cell inoculation. Two shell vials are inoculated in this manner and then incubated at 35 C, one for 24 hours and the other for 48 hours. After incubation they are observed for cytopathic effect (CPE) using standard immunofluorescent staining. CPE negative is considered to be an effective kill of HSV. CPE positive is considered to be a failure. Positive and negative controls are run with all experiments.

### EXAMPLE 1

### Formulation 1

The Formulation described in the present example contained the following components:

| Component | Mass (grams) |
|---|---|
| Water | 53.27 |
| Coco betaine - low | 42.8 |
| salt | |
| lauryl amine oxide | 1.33 |
| citric acid | 0.4 |
| Triclosan | 2.2 |

The total mass of the Formulation is 100 grams.

A 250 ml beaker equipped with a magnetic stir bar is charged with 53.27 grams water, 42.8 grams coco betaine—low salt, 1.33 g lauryl amine oxide and 0.4 g citric acid. The contents of the beaker are stirred and heated to 60° C. At 61° C. 2.2 grams of Triclosan are added with rapid stirring. In about 1-4 minutes a clear homogenous low viscosity solution forms. The solution is stirred at 61-63° C. for 20 minutes. The Formulation is cooled to room temperature and has a pH of about 4.6. One gram of the clear homogenous solution is added to 4 grams of water and a clear homogenous solution forms for a 1:5 dilution. An additional 5 grams of water is added to this clear solution and a clear homogenous solution forms for a 1:10 dilution. Sequential dilutions are continued up to 1:50 dilution with each dilution still giving a clear homogenous solution.

Testing of the Formulation shows that it has superior antimicrobial efficacy. In the zones of inhibition test, this Formulation caused a 51 millimeter Zone of Inhibition for Staphylococcus aureus and a 42 millimeter Zone of Inhibition for Escherichia coli and a 29 millimeter Zone of Inhibition for Enterococcus The Formulation also displayed bacteriocidal properties for both S. aureus and E. coli after a 10 minute exposure to the Formulations at all dilutions. In the Plate Cidality test there was no growth observed for these three microorganisms.

### EXAMPLE 2

### Formulation 2

A Formulation was prepared according to the method of Example 1, but contained the following components:

| Component | Mass (grams) |
|---|---|
| Water | 26.38 |
| Coco betaine - low | 21.40 |
| salt | |
| lauryl amine oxide | 0.67 |
| citric acid | 0.2 |
| Triclosan | 1.1 |
| Zonyl FS510 | 0.5 |

The total mass of the Formulation is 50.25 grams. Zonyl FS510 was added after the Triclosan. The initial Formulation was clear and the dilutions were also clear. 4

Testing of the Formulation shows that it has superior antimicrobial efficacy. In the zones of inhibition test, this Formulation caused a 53 millimeter Zone of Inhibition for Staphylococcus aureus and a 42 millimeter Zone of Inhibition for Escherichia coli.

### EXAMPLE 3

### Formulation 3

A Formulation was prepared according to the method of Example 1, but contained the following components:

| Component | Mass (grams) |
|---|---|
| Water | 22.49 |
| Coco betaine - low | 21.40 |
| salt | |
| lauryl amine oxide | 0.67 |
| citric acid | 0.2 |
| Triclosan | 1.1 |
| ammonium bifluoride | 1.2 |
| Phosphoric acid | 2.94 |

The total mass of the Formulation is 50 grams. The ammonium bifluoride and phosphoric acid were added to the Formulation after the Triclosan at approximately 60° C. The initial Formulation was clear and the dilutions were also clear.

Testing of the Formulation shows that it has superior antimicrobial efficacy. In the zones of inhibition test, this Formulation caused a 41 millimeter Zone of Inhibition for Staphylococcus aureus and a 30 millimeter Zone of Inhibition for Escherichia coli.

### EXAMPLE 4

### Formulation 4

A Formulation was prepared according to the method of Example 1, but contained the following components:

| Component | Mass (grams) |
|---|---|
| Water | 53.27 |
| Coco betaine - low | 42.8 |
| salt | |
| lauryl amine oxide | 1.33 |
| citric acid | 0.4 |
| Triclosan | 2.2 |

The total mass of the Formulation is 100 grams. The initial Formulation was clear and the dilutions were also clear.

Testing of the Formulation shows that it has superior antimicrobial efficacy. In the zones of inhibition test, this Formulation caused a 50 millimeter Zone of Inhibition for Staphylococcus aureus and a 39 millimeter Zone of Inhibition for Escherichia coli.

### EXAMPLE 5

### Formulation 5

A high solids Formulation was prepared according to the method of Example 1, but contained the following components:

| Component | Mass (grams) |
|---|---|
| Coco betaine - low | 42.8 |
| salt | |
| lauryl amine oxide | 1.33 |
| citric acid | 0.4 |
| Triclosan | 2.2 |

The total mass of the Formulation is 46.73 grams. The initial Formulation was clear and the dilutions were also clear.

Testing of the Formulation shows that it has superior antimicrobial efficacy. In the zones of inhibition test, this Formulation caused a 52 millimeter Zone of Inhibition for Staphylococcus aureus and a 50 millimeter Zone of Inhibition for Escherichia coli and a 31 millimeter Zone of Inhibition for Enterococcus The Formulation also displayed bacteriocidal properties for both S. aureus and E. coli after a 10 minute exposure to the Formulations at all dilutions. In the Plate Cidality test there was no growth observed for these three microorganisms.

### EXAMPLE 6

### Formulation 6

A high solids Formulation was prepared according to the method of Example 1, but contained the following components:

| Component | Mass (grams) |
|---|---|
| Octylbetaine | 25.0 |
| lauryl amine oxide | 2.09 |
| citric acid | 0.15 |
| sodium citrate | 0.41 |
| Triclosan | 1.1 |

The total mass of the Formulation is 28.75 grams. The initial Formulation was clear and the dilutions were also clear.

Testing of the Formulation shows that it has superior antimicrobial efficacy. In the zones of inhibition test, this Formulation caused a 45 millimeter Zone of Inhibition for Staphylococcus aureus and a 37 millimeter Zone of Inhibition for Escherichia coli and a 29 millimeter Zone of Inhibition for Enterococcus. The Formulation also displayed bacteriocidal properties for both S. aureus and E. coli. after a 10 minute exposure to the Formulations at all dilutions. In the Plate Cidality test there was no growth observed for these three microorganisms.

### EXAMPLE 7

### Formulation 7

A Formulation was prepared as described below and contained the following components:

| Component | Mass (grams) |
|---|---|
| Water | 11.63 |
| Cocobetaine - low | 21.40 |
| salt | |
| lauryl amine oxide | 0.67 |
| citric acid | 0.2 |
| Triclosan | 1.1 |
| Hydrogen peroxide | 15 |
| (30%) | |

The total mass of the Formulation is 50 grams. The initial Formulation was clear and the dilutions were also clear.

A 250 ml beaker equipped with a magnetic stir bar is charged with 11.63 grams water, 21.4 grams coco betaine—low salt, 0.66 grams lauryl amine oxide, and 0.2 grams citric acid. The contents of the beaker are stirred and heated to 60° C. At 61° C. 2.2 grams of Triclosan are added with rapid stirring. In about 1-4 minutes a clear homogenous low viscosity solution forms. The solution is stirred at 59-63° C. for 20 minutes. The Formulation is cooled to room temperature (22° C.) and 15 grams of hydrogen peroxide are added. The solution is stirred for 10 minutes. The clear homogenous low viscosity solution has a pH of about 4.8. One gram of the clear homogenous solution is added to 4 grams of water and a clear homogenous solution forms for a 1:5 dilution. An additional 5 grams of water is added to this clear solution and a clear homogenous solution forms for a 1:10 dilution. Sequential dilutions are continued up to 1:50 dilution with each dilution still giving a clear homogenous solution. Testing of the Formulation confirms this and shows it has superior antimicrobial efficacy.

Testing of the Formulation shows that it has superior antimicrobial efficacy. In the zones of inhibition test, this Formulation caused a 57 millimeter Zone of Inhibition for Staphylococcus aureus and a 43 millimeter Zone of Inhibition for Escherichia coli.

### EXAMPLE 8

### Formulation 8

A Formulation was prepared as described above in Example 7 and contained the following components:

| Component | Mass (grams) |
|---|---|
| Water | 20.2 |
| Cocobetaine - low | 21.40 |
| salt | |
| lauryl amine oxide | 0.67 |
| citric acid | 0.2 |
| Triclosan | 1.1 |
| t-butylhydroperoxide | 6.43 |
| (70%) | |

The total mass of the Formulation is 50 grams. The initial Formulation was mostly clear and the dilutions were also clear.

Testing of the Formulation shows that it has superior antimicrobial efficacy. In the zones of inhibition test, this Formulation caused a 112 millimeter Zone of Inhibition for Staphylococcus aureus and a 106 millimeter Zone of Inhibition for Escherichia coli.

### EXAMPLE 9

### Formulation 9

A Formulation was prepared as described above as in Example 7 and contained the following components:

| Component | Mass (grams) |
|---|---|
| Water | 44.67 |
| Cocobetaine - low | 42.8 |
| salt | |
| lauryl amine oxide | 1.33 |
| citric acid | 9.0 |
| Triclosan | 2.2 |

The total mass of the Formulation is 100 grams. The initial Formulation was clear and the dilutions were also clear.

Testing of the Formulation shows that it has superior antimicrobial efficacy. In the zones of inhibition test, this Formulation caused a 40.5 millimeter Zone of Inhibition for Staphylococcus aureus and a 33 millimeter Zone of Inhibition for Escherichia coli.

### EXAMPLE 10

### Formulation 10

A Formulation was prepared as described above as in Example 7. The Formulation contained the following components:

| Component | Mass (grams) |
|---|---|
| Water | 5.76 |
| Coco betaine - low | 21.4 |
| salt | |
| lauryl amine oxide | 1.0 |
| mysrtyl amine oxide | 1.0 |
| cocamidopropyl | 1.34 |
| amine oxide | |
| citric acid | 4.5 |
| Hydrogen peroxide | 15 |
| (30%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 61 millimeter Zone of Inhibition for Staphylococcus aureus and a 45 millimeter Zone of Inhibition for Escherichia coli and a 29 millimeter Zone of Inhibition for Pseudomonas aeruginosa. In the Plate Cidality test there was no growth observed for these three microorganisms.

### EXAMPLE 11

### Formulation 11

A Formulation was prepared as described above as in Example 7. The Formulation contained the following components:

| Component | Mass (grams) |
|---|---|
| Water | 7.0 |
| Coco betaine - low | 21.40 |
| salt | |
| lauryl amine oxide | 1.0 |
| citric acid | 4.5 |
| Triclosan | 1.1 |
| Hydrogen peroxide | 15 |
| (30%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 60.5 millimeter Zone of Inhibition for Staphylococcus aureus and a 44 millimeter Zone of Inhibition for Escherichia coli and a 29 millimeter Zone of Inhibition for Pseudomonas aeruginosa. In the Plate Cidality test there was no growth observed for these three microorganisms.

### EXAMPLE 12

### Formulation 12

A Formulation was prepared as described above as in Example 7. The Formulation contained the following components:

| Component | Mass (grams) |
|---|---|
| Water | 20.76 |
| Coco betaine - low | 21.4 |
| salt | |
| lauryl amine oxide | 1.0 |
| myristyl amine oxide | 1.0 |
| cocamidopropyl | 1.34 |
| amine oxide | |
| citric acid | 4.5 |

The total mass of the Formulation is 50 grams. The initial Formulation was clear and the dilutions were also clear.

Testing of the Formulation shows that it has superior antimicrobial efficacy. In the zones of inhibition test, this Formulation caused a 24 millimeter Zone of Inhibition for Staphylococcus aureus and a 19 millimeter Zone of Inhibition for Escherichia coli.

### EXAMPLE 13

### Formulation 13

A Formulation was prepared as described above as in Example 7. The Formulation contained the following components:

| Component | Mass (grams) |
|---|---|
| Water | 4.66 |
| Coco betaine - low | 21.4 |
| salt | |
| lauryl amine oxide | 1.0 |
| myristyl amine oxide | 1.0 |
| cocamidopropyl | 1.34 |
| amine oxide | |
| citric acid | 4.5 |
| Triclosan | 1.1 |
| Hydrogen peroxide | 15 |
| (30%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 61 millimeter Zone of Inhibition for Staphylococcus aureus and a 44 millimeter Zone of Inhibition for Escherichia coli and a 31 millimeter Zone of Inhibition for Pseudomonas aeruginosa. In the Plate Cidality test there was no growth observed for these three microorganisms.

### EXAMPLE 14

### Formulation 14

A Formulation was prepared as described above as in Example 7. The Formulation contained the following components:

| Component | Mass (grams) |
|---|---|
| Water | 5.92 |
| Cocobetaine - low | 35.68 |
| salt | |
| lauryl amine oxide | 0.67 |
| citric acid | 0.2 |
| Triclosan | 1.1 |
| t-butylhydroperoxide | 6.43 |
| (70%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 110 millimeter Zone of Inhibition for Staphylococcus aureus and a 110 millimeter Zone of Inhibition for Escherichia coli and a 70 millimeter Zone of Inhibition for Pseudomonas aeruginosa and a 36 millimeter Zone of Inhibition for Candida albicans and a 49 millimeter Zone of Inhibition for Aspergillus niger and a 28 millimeter Zone of Inhibition for Mycobacterium fortuitum and a 74 millimeter Zone of Inhibition for methicillin resistant Staphylococcus aureus and a Zone of Inhibition of 35 millimeters for vancomycin-resistant Enterococci and a Zone of Inhibition of 100 millimeters for mulit-drug resistant Pseudomonas aeruginosa. In the Plate Cidality test there was no growth observed for all these nine microorganisms. Herpes Simplex cultures were tested against this formulation after exposing it to this formulation for 15 minutes before being cultured and then subjected to the shell vial culturing methodology. There was no growth observed after 24 and 48 hours with this formulation while good growth was observed in the controls after both 24 and 48 hours.

### EXAMPLE 15

### Formulation 15

A Formulation was prepared as described above as in Example 7. The Formulation contained the following components:

| Component | Mass (grams) |
|---|---|
| Water | 16.71 |
| Cocobetaine - low | 25.0 |
| salt | |
| lauryl amine oxide | 0.69 |
| citric acid | 0.22 |
| Triclosan | 1.1 |
| t-butylhydroperoxide | 6.42 |
| (70%) | |

The total mass of the Formulation is 50.14 grams. The initial Formulation was clear and the dilutions were also clear.

Testing of the Formulation shows that it has superior antimicrobial efficacy. In the zones of inhibition test, this Formulation caused a 100 millimeter Zone of Inhibition for Staphylococcus aureus and a 110 millimeter Zone of Inhibition for Escherichia coli and a 48 millimeter Zone of Inhibition for Pseudomonas aeruginosa.

### EXAMPLE 16

### Formulation 16

A Formulation was prepared as described above as in Example 7. The Formulation contained the following components:

| Component | Mass (grams) |
|---|---|
| Water | 13.77 |
| Cocobetaine - low | 21.4 |
| salt | |
| lauryl amine oxide | 7.1 |
| citric acid | 0.2 |
| Triclosan | 1.1 |
| t-butylhydroperoxide | 6.43 |
| (70%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 102 millimeter Zone of Inhibition for Staphylococcus aureus and a 100 millimeter Zone of Inhibition for Escherichia coli and a 52 millimeter Zone of Inhibition for Pseudomonas aeruginosa. In the Plate Cidality test there was no growth observed for these three microorganisms.

### EXAMPLE 17

### Formulation 17

A Formulation was prepared as described above as in Example 7. The Formulation contained the following components:

| Component | Mass (grams) |
|---|---|
| Water | 22.35 |
| Cocobetaine - low | 23.54 |
| salt | |
| lauryl amine oxide | 0.67 |
| citric acid | 0.2 |
| Triclosan | 1.1 |
| t-butylhydroperoxide | 2.14 |

The total mass of the Formulation is 50 grams. The initial Formulation was clear and the dilutions were also clear.

Testing of the Formulation shows that it has superior antimicrobial efficacy. In the zones of inhibition test, this Formulation caused a 60 millimeter Zone of Inhibition for Staphylococcus aureus and a 56 millimeter Zone of Inhibition for Escherichia coli and a 41 millimeter Zone of Inhibition for Pseudomonas aeruginosa. In the Plate Cidality test there was no growth observed for these three microorganisms.

### EXAMPLE 18

### Formulation 18

A Formulation was prepared as described above as in Example 7. The Formulation contained the following components:

| Component | Mass (grams) |
|---|---|
| Water | 13.06 |
| Cocobetaine - low salt | 28.54 |
| Lauryl amine oxide | 0.67 |
| citric acid | 0.2 |
| Triclosan | 1.1 |
| t-butylhydroperoxide | 6.43 |
| (70%) | |

The total mass of the Formulation is 50 grams. The initial Formulation was clear and the dilutions were also clear.

Testing of the Formulation shows that it has superior antimicrobial efficacy. In the zones of inhibition test, this Formulation caused a 110 millimeter Zone of Inhibition for Staphylococcus aureus and a 110 millimeter Zone of Inhibition for Escherichia coli and a 70 millimeter Zone of Inhibition for Pseudomonas aeruginosa. In the Plate Cidality test there was no growth observed for these three microorganisms.

### EXAMPLE 19

### Formulation 19

| Component | Mass (grams) |
|---|---|
| Water | 21.30 |
| Lauryl amine oxide | 0.67 |
| cocobetaine | 21.40 |
| citric acid | 0.2 |
| t-butylhydroperoxide | 6.43 |

The total mass of the Formulation is 50 grams.

Formulation 19 was prepared by sequentially mixing the components in the order listed at room temperature. Total mixing time at room temperature was about 25 minutes.

Testing of the Formulation shows that it has superior antimicrobial efficacy. In the zones of inhibition test, this Formulation caused a 98 millimeter Zone of Inhibition for Staphylococcus aureus and an 85 millimeter Zone of Inhibition for Escherichia coli and a 55 millimeter Zone of Inhibition for Pseudomonas aeruginosa. In the Plate Cidality test there was no growth observed for these three microorganisms.

### EXAMPLE 20

### Formulation 20

This example demonstrates that by selecting a liquid antimicrobial agent that can dissolve or liquefy the second solid antimicrobial agent, it is not necessary employ heat in the Formulation process to give a clear, homogeneous Formulation which is also clear and homogeneous upon sequential dilution. These clear and homogeneous Formulations have a high degree of antimicrobial efficacy. The Formulation was prepared as described below and contained the following components:

| Component | Mass (grams |
|---|---|
| t-butyl hydroperoxide (70%) | 12.86 |
| Triclosan | 2.20 |
| Cocobetaine - low salt | 71.36 |
| Citric acid | 0.40 |
| Aqueous 2000 ppm iron | 2.00 |
| sulfate solution | |
| Water | 11.18 |

The total mass of the Formulation is 100 grams. The initial Formulation was clear and homogeneous and the dilutions were also clear and homogeneous.

The following procedure was used to prepare this Formulation:

A 250 ml beaker equipped with a magnetic stir bar was charged with 12.86 grams of t-butyl hydroperoxide (70%) and 2.20 grams of Triclosan. The solid—liquid dispersion was stirred at room temperature. After about 10 to 15 minutes of stirring an all liquid mixture forms. To the two phase oil and water mixture is added 71.36 grams of cocobetaine—low salt version—and stirred at room temperature. After about 2-5 minutes a clear and homogeneous solution forms. To the solution is added 0.40 grams of citric acid and stirring is continued at room temperature for 30 minutes. To the clear and homogeneous solution are added 2.00 grams of an aqueous solution containing 2000 ppm of iron sulfate followed by 11.18 grams of water. The solution is stirred an additional 10 minutes. The clear homogeneous solution has a low viscosity and a pH of 6.5. One gram of the clear homogeneous solution is added to 4 grams of water and a clear homogeneous solution forms for a 1:5 dilution. An additional 5 grams of water are added to this clear solution and a clear homogeneous solution forms for a 1:10 dilution. Sequential dilutions are continued up to 1:50 dilution with each dilution giving a clear homogeneous solution.

### EXAMPLE 21

Two Herpes Simplex ATCC (#vr-733) cultures were tested against Formulation 14 for (eliminate: 12 for) 15 minutes before being cultured) and then subjected to shell vial culturing as outlined in methodology below. The result was that each was CPE negative in that no growth in either culture after exposure to Formulation 14 after 24 and 48 hours, was observed while good growth was observed in controls after both 24 and 48 hours.”

Examples 22-43 were prepared using the following procedure:

A 250 ml beaker equipped with a magnetic stir bar is charged with water (if used in the formulation), surfactant, citric acid, sodium citrate (if used in the formulation) and a fluorocarbon wetting agent (if used in the formulation. The contents of the beaker are stirred and heated to about 60-63° C. At about 60° C. Triclosan is added with rapid stirring. In about 1-4 minutes a clear homogenous low viscosity solution forms. The solution is stirred at about 60-63° C. for 20 minutes. The Formulation is cooled to room temperature and has a pH in the range of about 4.0-7.0.

### EXAMPLE 22

Example 22 contained the following components:

| Component | Mass (grams) |
|---|---|
| Water | 0.00 |
| Coco betaine - low | 22.3 |
| salt | |
| lauryl amine oxide | 2.1 |
| citric acid | 0.15 |
| Sodium citrate | 0.41 |
| Triclosan | 1.0 |

The total mass of the Formulation is 25.96 grams and a pH of 5.5.

### EXAMPLE 23

Example 23 contained the following components:

| Component | Mass (grams) |
|---|---|
| Water | 54.6 |
| Coco betaine - low | 42.8 |
| salt | |
| citric acid | 0.40 |
| Triclosan | 2.2 |

The total mass of the Formulation is 100.0 grams and a pH of 4.5.

### EXAMPLE 24

Example 24 contained the following components:

| Component | Mass (grams) |
|---|---|
| Water | 0.00 |
| Coco betaine - low | 28.6 |
| salt | |
| Lauryl amine oxide | 1.33 |
| citric acid | 0.40 |
| Triclosan | 1.6 |

The total mass of the Formulation is 31.93 grams and a pH of 4.7.

### EXAMPLE 25

Example 25 contained the following components:

| Component | Mass (grams) |
|---|---|
| Water | 64.99 |
| Coco betaine - low | 31.43 |
| salt | |
| Lauryl amine oxide | 0.98 |
| citric acid | 0.40 |
| Triclosan | 2.2 |

The total mass of the Formulation is 100.0 grams and a pH of 4.3.

### EXAMPLE 26

Example 26 contained the following components:

| Component | Mass (grams) |
|---|---|
| Water | 69.04 |
| Coco betaine - low | 27.5 |
| salt | |
| Lauryl amine oxide | 0.86 |
| citric acid | 0.40 |
| Triclosan | 2.2 |

The total mass of the Formulation is 100.0 grams and a pH of 4.2.

### EXAMPLE 27

Example 27 contained the following components:

| Component | Mass (grams) |
|---|---|
| Water | 36.69 |
| Coco betaine - low | 11.77 |
| salt | |
| Lauryl amine oxide | 0.36 |
| citric acid | 0.21 |
| Triclosan | 1.1 |

The total mass of the Formulation is 50.13 grams and a pH of 4.2.

### EXAMPLE 28

Example 28 contained the following components:

| Component | Mass (grams) |
|---|---|
| Water | 65.9 |
| Coco betaine - low | 26.6 |
| salt | |
| Triclosan | 7.5 |

The total mass of the Formulation is 100.0 grams.

### EXAMPLE 29

Example 29 contained the following components:

| Component | Mass (grams) |
|---|---|
| Water | 56.07 |
| Cocamidopropyl | 40.0 |
| betaine | |
| Lauryl amine oxide | 1.33 |
| citric acid | 0.40 |
| Triclosan | 2.2 |

The total mass of the Formulation is 100.0 grams and a pH of 4.4.

### EXAMPLE 30

Example 30 contained the following components:

| Component | Mass (grams) |
|---|---|
| Water | 57.4 |
| Cocamidopropyl | 40.0 |
| betaine | |
| citric acid | 0.40 |
| Triclosan | 2.2 |

The total mass of the Formulation is 100.0 grams and a pH of 4.0.

### EXAMPLE 31

Example 31 contained the following components:

| Component | Mass (grams) |
|---|---|
| Water | 64.4 |
| Cocamidopropyl | 33.0 |
| betaine | |
| citric acid | 0.40 |
| Triclosan | 2.2 |

The total mass of the Formulation is 100.0 grams and a pH of 4.0.

### EXAMPLE 32

Example 32 contained the following components:

| Component | Mass (grams) |
|---|---|
| Water | 68.07 |
| Cocamidopropyl | 29.33 |
| betaine | |
| citric acid | 0.40 |
| Triclosan | 2.2 |

The total mass of the Formulation is 100.0 grams and a pH of 4.0.

### EXAMPLE 33

Example 33 contained the following components:

| Component | Mass (grams) |
|---|---|
| Water | 71.73 |
| Cocamidopropyl | 25.67 |
| betaine | |
| citric acid | 0.40 |
| Triclosan | 2.2 |

The total mass of the Formulation is 100.0 grams and a pH of 4.0.

### EXAMPLE 34

Example 34 contained the following components:

| Component | Mass (grams) |
|---|---|
| Water | 0.00 |
| Octyl betaine | 22.5 |
| Lauryl amine oxide | 7.5 |
| citric acid | 0.15 |
| Sodium citrate | 0.41 |
| Triclosan | 1.2 |

The total mass of the Formulation is 31.76 grams and a pH of 6.3. The antimicrobial effectiveness for this composition is shown in Table 3 hereinabove.

### EXAMPLE 35

Example 35 contained the following components:

| Component | Mass (grams) |
|---|---|
| Water | 17.12 |
| Octyl betaine | 22.5 |
| Lauryl amine oxide | 7.5 |
| citric acid | 0.3 |
| Sodium citrate | 0.83 |
| Triclosan | 1.5 |

The total mass of the Formulation is 49.75 grams and a pH of 6.0. The antimicrobial effectiveness for this composition is shown in Table 3 hereinabove.

### EXAMPLE 36

Example 36 contained the following components:

| Component | Mass (grams) |
|---|---|
| Water | 18.47 |
| Octyl betaine | 25.0 |
| Lauryl amine oxide | 4.17 |
| citric acid | 0.15 |
| Sodium citrate | 0.41 |
| Triclosan | 1.8 |

The total mass of the Formulation is 50.0 grams and a pH of 6.5.

### EXAMPLE 37

Example 37 contained the following components:

| Component | Mass (grams) |
|---|---|
| Water | 35.87 |
| Octyl betaine | 9.0 |
| citric acid | 0.3 |
| Sodium citrate | 0.83 |
| Zonyl ® FSN 100 | 0.25 |
| Triclosan | 3.75 |

The total mass of the Formulation is 50.0 grams and a pH of 5.0. Zonyl® FSN 100 is a fluorocarbon wetting agent available from DuPont.

### EXAMPLE 38

Example 38 contained the following components:

| Component | Mass (grams) |
|---|---|
| Water | 19.54 |
| Lauryl amine oxide | 29.16 |
| citric acid | 0.2 |
| Triclosan | 1.1 |

The total mass of the Formulation is 50.0 grams and a pH of 6.0.

### EXAMPLE 39

Example 39 contained the following components:

| Component | Mass (grams) |
|---|---|
| Water | 64.25 |
| Lauryl amine oxide | 30.0 |
| citric acid | 0.6 |
| Sodium citrate | 1.65 |
| Zonyl ® FSN 100 | 0.5 |
| Triclosan | 1.5 |
| Ammonium bifluoride | 1.5 |

The total mass of the Formulation is 100.0 grams and a pH of 4.7. The antimicrobial effectiveness for this composition is shown in Table 3 hereinabove.

### EXAMPLE 40

Example 40 contained the following components:

| Component | Mass (grams) |
|---|---|
| Water | 2.5 |
| Lauryl amine oxide | 30.0 |
| citric acid | 0.6 |
| Sodium citrate | 1.65 |
| Zonyl ® FSN 100 | 0.5 |
| Triclosan | 7.5 |

The total mass of the Formulation is 42.75 grams and a pH of 6.1.

### EXAMPLE 41

Example 41 contained the following components:

| Component | Mass (grams) |
|---|---|
| Water | 3.31 |
| lauryl betaine | 42.9 |
| Lauryl amine oxide | 1.33 |
| citric acid | 0.15 |
| Sodium citrate | 0.41 |
| Triclosan | 1.9 |

The total mass of the Formulation is 50.0 grams and a pH of 5.5.

### EXAMPLE 42

Example 42 contained the following components:

| Component | Mass (grams) |
|---|---|
| Water | 23.14 |
| lauryl betaine | 22.3 |
| Lauryl amine oxide | 2.1 |
| citric acid | 0.15 |
| Sodium citrate | 0.41 |
| Triclosan | 1.9 |

The total mass of the Formulation is 50.0 grams and a pH of 6.0.

### EXAMPLE 43

Example 43 contained the following components:

| Component | Mass (grams) |
|---|---|
| Water | 0.00 |
| octyl amine oxide | 25.00 |
| citric acid | 0.15 |
| Sodium citrate | 0.41 |
| Triclosan | 0.9 |

The total mass of the Formulation is 26.46 grams and a pH of 7.0. The antimicrobial effectiveness for this composition is shown in Table 3 hereinabove.

Examples 44-46 were prepared using the following procedure:

A 250 ml beaker equipped with a magnetic stir bar is charged with water, surfactant, citric acid, and sodium citrate (if used in the formulation). At room temperature the contents of the beaker are stirred while the components are sequentially added at about one minute apart. Upon completion of the addition of the components the formulation is stirred at room temperature for about 30 minutes.

### EXAMPLE 44

Example 44 contained the following components:

| Component | Mass (grams) |
|---|---|
| Water | 66.05 |
| cocamidopropyl | 15.0 |
| betaine | |
| myristamineamine | 15.0 |
| oxide | |
| citric acid | 0.8 |
| Sodium citrate | 1.65 |
| Triclosan | 1.5 |

The total mass of the Formulation is 100.0 grams and a pH of 5.5. The antimicrobial effectiveness for this composition is shown in Table 3 hereinabove.

### EXAMPLE 45

Example 45 contained the following components:

| Component | Mass (grams) |
|---|---|
| Water | 53.27 |
| cocobetaine - low | 42.8 |
| salt version | |
| Lauryl amine oxide | 1.33 |
| citric acid | 0.4 |
| Triclosan | 2.2 |

The total mass of the Formulation is 100.0 grams and a pH of 4.7. The antimicrobial effectiveness for this composition is shown in Table 3 hereinabove.

### EXAMPLE 46

Example 46 contained the following components:

| Component | Mass (grams) |
|---|---|
| Water | 56.75 |
| Lauryl amine oxide | 30.0 |
| citric acid | 3.0 |
| Sodium citrate | 8.25 |
| Zonyl ® FSN 100 | 0.5 |
| Triclosan | 1.5 |

The total mass of the Formulation is 100.0 grams and a pH of 6.0.

### EXAMPLE 47

Example 47 contained the following components:

| Component | Mass (grams) |
|---|---|
| Water | 11.63 |
| cocobetaine - low | 21.4 |
| salt version | |
| Lauryl amine oxide | 0.67 |
| citric acid | 0.2 |
| Triclosan | 1.1 |
| Peracetic acid - 30% | 15.0 |

The total mass of the Formulation is 50.0 grams and a pH of 3.0.

Example 47 was prepared using the following procedure:

A 250 ml beaker equipped with a magnetic stir bar is charged with 11.63 grams of water, 21.4 grams of cocobetaine—low salt version, 0.67 grams of lauryl amine oxide, 0.2 grams of citric acid, The contents of the beaker are stirred and heated to about 60-63° C. At about 60° C. 1.1 grams of Triclosan are added with rapid stirring. In about 1-4 minutes a clear homogenous low viscosity solution forms. The solution is stirred at about 60-63° C. for 20 minutes. The Formulation is cooled to room temperature and 15.0 grams of 30% peracetic acid are added with stirring. The solution is stirred an additional 10 minutes at room temperature. The formulation has a pH of about 3.0. The formulation is initially clear and homogeneous and remains clear and homogeneous upon sequential dilution.

### EXAMPLE 48

Example 48 demonstrates an ultra high % active ingredient and % solids antimicrobial formulation. Example 48 contained the following components:

| | Mass |
|---|---|
| Component | (grams) |
| Lauramidopropyl | 12.48 |
| betaine - powder | |
| t-butyl hydroperoxide | 3.10 |

Formulation 48 was prepared by the following procedure:

To a beaker containing 12.48 grams of powder lauramidopropyl betaine (98%) was added 3.10 grams of t-butyl hydroperoxide (70%). The contents of the beaker were manually stirred with a spatula at room temperature for 5 minutes to a uniform and homogeneous paste. The formulation has a % active ingredient of 92.4% and a % solid of 78.5%. By reducing the amount of t-butyl hydroperoxide to 1.00 grams a 97.8% active ingredient and 90.7% solids formulation can be prepared.

The 98% powder lauramidopropyl betaine is available from the Mcintyre Group Ltd. as Macham® 1200.

The other surfactants used in the above examples are available from the following commercial sources:
Cocobetaine, low salt version, from Mcintyre Group Ltd as Macham® CB35ULSHP, 28%
Lauryl amine oxide from Stepan Company as Ammonyx® LO, 30%
Cocamidopropyl betaine from Stepan Company as Amphosol® CA. 30%
Octyl betaine from Mcintyre Group Ltd as Macham® OCTLS, 50%
Lauryl betaine from Mcintyre Group Ltd as Macham® LB35, 28%
Octyl amine oxide from Lonza Inc. as FMB AO-8, 40%
Myristyl amine oxide from Stepan Company as Ammonyx® MO, 30%
Fluorocarbon wetting agents Zonyl® FSN 100 and Zonyl® FS 510 from Dupont.

### EXAMPLE 49

This example illustrates an ultra high solids Antimicrobial Formulation

The formulation contained the following components:

| Component | Mass (grams) |
|---|---|
| Cocobetaine - solid 98% | 5.03 |
| Triclosan | 1.12 |

Total mass of the formulation is 6.15 grams.

An aluminum weighing pan of approximately 2 inches in diameter containing 5.03 grams of solid, brittle waxy cocobetaine was place on a hot plate set at 100° C. On top of the cocobetaine solid was placed 1.12 grams of Triclosan. As the aluminum pan was heated the Triclosan melted and softened the cocobetaine. Once the Triclosan was completely melted the mixture was stirred while hot for 5 minutes with a spatula. A homogeneous off white paste formed which remained as a paste when cooled to room temperature. The off white paste had a solids of about 98.4% with about 18.2% Triclosan.

The solid cocobetaine was obtained from the commercially available aqueous solution (Macham CB35ULSHP) by air drying 14.37 grams of the aqueous solution in an aluminum weighing pan for 5 days at room temperature in a fume hood. The aluminum weighing pan containing the air dried cocobetaine was placed on a hot plate set at 100° C. for 5 minutes during which time a constant mass was achieved. The solid brittle/waxy cocobetaine was used in the above formulation.

### EXAMPLE 50

### Formulation 50

This example illustrates an antimicrobial Formulation with a High Level of t-butyl Hydroperoxide with Iron Sulfate Present.

The first component of the formulation was prepared as described above as in Example 7.

The formulation contained the following two components:

| Component | Mass (grams) |
|---|---|
| Water | 3.27 |
| Cocobetaine - low salt | 57.16 |
| version | |
| Lauryl amine oxide | 1.34 |
| Citric acid | 0.40 |
| Triclosan | 2.20 |
| Mixture of cocobetaine and | 14.20 cocobetaine |
| t-butyl hydroperoxide | 21.43 t-butyl hydroperoxide |

Total mass of Component 1 is 100 grams.

Component 2 consists of 20 grams of aqueous iron sulfate with an iron sulfate level of 20 ppm.

The final step in preparing this formulation consisted of taking a 10 gram portion of Component 1 and adding it to Component 2, the 20 grams of iron sulfate solution, and stirring at room temperature for 10 minutes.

Testing of the formulation this formulation shows that it has superior antimicrobial efficacy. In the Zone of Inhibition test, this formulation caused a 60 millimeter Zone of Inhibition for Staphylococcus aureus and a 64 millimeter Zone of Inhibition for Escherichia coli. In the Plate Cidality test there was no growth observed for either of these organisms. Additionally, the Formulation exhibited Zones of Inhibition of 40 mm for Candida albicans and 52 mm for Aspergillus niger.

### EXAMPLE 51

This example illustrates activity against spore-forming organisms.

Two difficult to kill spore forming microorganisms, Bacillus anthracis and Bacillus subtilus were challenged with the Formulation 14 in which one part of the Formulation 14 was diluted with two parts of water containing 20 ppm iron sulfate.

In the Zone of Inhibition test, a 79 mm zone of Inhibition was Observed with Bacillus subtilus and an 80 mm zone of inhibition with Bacillus anthracis. In the Plate Cidality test there was no growth observed for both of these microorganisms.

Formulation 14 was tested in a time to kill study with about an 8% spore population) bacteria. Approximately 1.0 ml of Formulation 14 were used in the study into which 10/8 cfu/ml was added and viable counts were measured over time intervals. The time intervals were 1, 5, 15, 30 minutes and 1, 2, 4, 6, 8, 12, 24 hours. Time intervals might be slightly different for different runs. At the designated time intervals about 1/1000 of an ml of solution was subcultured onto Petri dishes containing TSA (trypticase soy agar) with 5% Sheep Blood. The plates were incubated for about 24 hours and the number of colonies were counted if the counts were less than 300, otherwise counts were recorded as TNTC (too numerous to count). The results were recorded and plotted.

The results were as follows:

| Time | | Remaining Colonies |
|---|---|---|
| 1 | min | 2 |
| 5 | min | 2 |
| 10 | min | 1 |
| 30 | min | 0 |
| 1 | hour | 1 |
| 2 | hours | 0 |
| 3 | hours | 0 |
| 4 | hours | 0 |
| 6 | hours | 0 |
| 12 | hours | 1 |
| 24 | hours | 1 |

Blank control Heavy growth, too numerous to count

### EXAMPLE 52

The following is an example of an unstable peracetic acid containing antimicrobial formulation (Formulation 52).

Formulation 52 contained the following components:

| Component | Mass (grams) |
|---|---|
| Water | 11.63 |
| cocobetaine - low | 21.4 |
| salt version | |
| Lauryl amine oxide | 0.67 |
| citric acid | 0.2 |
| Triclosan | 1.1 |
| Peracetic acid - 30% | 15.0 |

Formulation 52 was prepared as follows:

A 250 ml beaker equipped with a magnetic stir bar and thermometer is charged with 11.63 grams of water, 21.4 grams of cocobetaine—low salt version, 0.67 grams of lauryl amine oxide, 0.2 grams of citric acid, The contents of the beaker are stirred and heated to about 60-63° C. At about 60° C. 1.10 grams of Triclosan are added with rapid stirring. In about 1-4 minutes a clear homogenous low viscosity solution forms. The solution is stirred at about 60-63° C. for 20 minutes. The formulation is cooled to room temperature and 15.0 grams of 30% peracetic acid are added with stirring. The solution is stirred an additional 10 minutes at room temperature. The formulation has a pH of about 3.0. The formulation is initially clear and homogeneous and remains clear and homogeneous upon sequential dilution.

This formulation began to degas almost immediately after preparation and after four months at room temperature continued to degas. Pressure in the storage bottle was relieved by loosening the cap on a weekly basis. Similar unstable formulations stored in plastic bottles which were not pressure relieved, bulged the plastic bottle considerably or ruptured the plastic bottles in less than two weeks at room temperature. This degassing of the formulation is an indication that the concentration of the active peroxy antimicrobial agent is constantly decreasing. Formulations with constantly changing concentrations of active ingredients have limited utility and applicability as an antimicrobial agent.

### EXAMPLE 53

A peroxy formulation (Formulation 53) which is stable and efficacious contained the following components:

| | |
|---|---|
| Component | Mass (grams) |

| | |
|---|---|
| Hydrogen peroxide 3% | 5.92 |
| stabilized aqueous | |
| solution¹ | |
| cocobetaine - low salt | 21.40 |
| version | |
| Lauryl amine oxide | 0.67 |
| citric acid | 0.20 |
| Triclosan | 1.1 |
| t-butyl hydroperoxide 70% | 6.43 |
| Peracetic acid - 30% | 14.28 |

Aqueous hydrogen peroxide solutions are commonly stabilized with 25-250 ppm of colloidal stannate, sodium pyrophosphate, acetanilide or Dequest® organophosphates from Monsanto.

The total mass of the formulation is 50.0 grams and has a pH of about 3.0 and a % solids of about 15%.

The above formulation is diluted at room temperature by either of two ways before use:
a. 1 part of the above formulation with 2 parts of 3% aqueous hydrogen peroxide stabilized
b. 1 part of the above formulation with 2 parts of water containing 20 ppm iron sulfate

The formulation diluted as described for part a. above was tested for antimicrobial efficacy. Testing of this formulation shows that it has superior antimicrobial efficacy. In the Zone of Inhibition test, this formulation caused a 66 millimeter Zone of Inhibition for Staphylococcus aureus and a 52 millimeter Zone of Inhibition for Escherichia coli and a 84 millimeter Zone of Inhibition for Bacillus anthracis.

In the Plate Cidality test there was no growth observed for any of these organisms.

Formulation 53 was prepared using the following procedure:

A 250 ml beaker equipped with a magnetic stir bar and thermometer is charged with 5.92 grams of 3% stabilized aqueous hydrogen peroxide, 21.40 grams of cocobetaine—low salt version, 0.67 grams of lauryl amine oxide, 0.2 grams of citric acid, The contents of the beaker are stirred and heated to about 60-63° C. The contents of the beaker were held at 60-63° C. for 10 minutes with stirring. After the 10 minute hold, at about 60° C. 1.10 grams of Triclosan are added with rapid stirring. In about 1-4 minutes a clear homogenous low viscosity solution forms. The solution is stirred at about 60-63° C. for 10 minutes. The formulation is cooled to room temperature and 6.43 g of 70% t-butyl hydroperoxide are added and stirred for about 1 minute to form a clear and homogeneous solution. Then 14.28 grams of 30% peracetic acid are added with stirring at room temperature. The solution is stirred an additional 10 minutes at room temperature. The formulation has a pH of about 3.0. The formulation is initially clear and homogeneous and remains clear and homogeneous upon sequential dilution. The above formulation and the two dilutions all were stable and showed no signs of degassing. After one month at room temperature none of the formulations showed any signs of degassing.

Formulations 54, 55 and 56 were prepared by the same procedure as in Formulation 53.

### EXAMPLE 54

A peroxy formulation (Formulation 54) which is stable and efficacious contained the following components:

| Component | Mass (grams) |
|---|---|
| Hydrogen peroxide 3% | 5.92 |
| stabilized aqueous solution | |
| cocobetaine - low salt | 34.18 |
| version | |
| Lauryl amine oxide | 0.67 |
| citric acid | 0.20 |
| Triclosan | 1.10 |
| t-butyl hydroperoxide 70% | 6.43 |
| Peracetic acid - 30% | 1.50 |

The total mass of the above formulation is 50.0 grams and has a pH of about 4.5 and a % solids of about 23%.

The above formulation is diluted at room temperature by either of two ways before use:
a. 1 part of the above formulation with 2 parts of 3% aqueous hydrogen peroxide stabilized
b. 1 part of the above formulation with 2 parts of water containing 20 ppm iron sulfate

The formulation is initially clear and homogeneous and remains clear and homogeneous upon sequential dilution. The above formulation and the two dilutions all were stable and showed no signs of degassing. After one month at room temperature none of the formulations showed any signs of degassing.

### EXAMPLE 55

A peroxy formulation (Formulation 55) which is stable and efficacious contained the following components:

| Component | Mass (grams) |
|---|---|
| Hydrogen peroxide 3% | 5.92 |
| stabilized aqueous solution | |
| cocobetaine - low salt | 21.40 |
| version | |
| Lauryl amine oxide | 0.67 |
| citric acid | 0.20 |
| Triclosan | 1.10 |
| t-butyl hydroperoxide 70% | 6.43 |
| Peracetic acid - 30% | 5.00 |

The total mass of the above formulation is 40.72 grams and has a pH of about 3.5 and a % solids of about 15%.

The above formulation is diluted at room temperature as follows before use:
1 part of the above formulation with 2 parts of 3% aqueous hydrogen peroxide stabilized

The formulation is initially clear and homogeneous and remains clear and homogeneous upon sequential dilution. The above formulation and the dilution all were stable and showed no signs of degassing.

Testing of this formulation shows that it has superior antimicrobial efficacy. In the Zone of Inhibition test, this formulation caused a 80 millimeter Zone of Inhibition for Staphylococcus aureus and a 101 millimeter Zone of Inhibition for Escherichia coli and a 100 millimeter Zone of Inhibition for Bacillus anthracis.

In the Plate Cidality test there was no growth observed for any of these organisms.

This formulation was tested in a time to kill study with about a 7% spore population bacteria. Approximately 1.0 ml of Formulation 14 were used in the study into which 10/8 cfu/ml was added and viable counts were measured over time intervals. The time intervals were 1, 5, 15, 30 minutes and 1, 2, 4, 6, 8, 12, 24 hours. Time intervals might be slightly different for different runs. At the designated time intervals about 1/1000 of an ml of solution was subcultured onto Petri dishes containing TSAR (triplicate soy agar) with 5% Sheep Blood. The plates were incubated for about 24 hours and the number of colonies were counted if the counts were less than 300, otherwise counts were recorded as TNTC (too numerous to count). The results were recorded.

The results were as follows:

| Time | | Remaining Colonies |
|---|---|---|
| 1 | min | 0 |
| 5 | min | 0 |
| 10 | min | 0 |
| 30 | min | 0 |
| 1 | hour | 0 |
| 2 | hours | 0 |
| 3 | hours | 0 |
| 4 | hours | 0 |
| 6 | hours | 0 |
| 12 | hours | 0 |
| 24 | hours | 0 |

Blank control Heavy growth, too numerous to count

### EXAMPLE 56

A peroxy formulation (Formulation 56) which is stable and efficacious contained the following components:

| Component | Mass (grams) |
|---|---|
| Hydrogen peroxide 3% | 10.20 |
| stabilized aqueous solution | |
| cocobetaine - low salt | 21.40 |
| version | |
| Lauryl amine oxide | 0.67 |
| citric acid | 0.20 |
| Triclosan | 1.10 |
| t-butyl hydroperoxide 70% | 6.43 |
| Peracetic acid - 30% | 10.00 |

The total mass of the above formulation is 50.00 grams and has a pH of about 3.0 and a % solids of about 18%.

The above formulation is diluted at room temperature as follows before use:
1 part of the above formulation with 2 parts of 3% aqueous hydrogen peroxide stabilized

The formulation is initially clear and homogeneous and remains clear and homogeneous upon sequential dilution. The above formulation and the dilution all were stable and showed no signs of degassing.

Testing of this formulation shows that it has superior antimicrobial efficacy. In the Zone of Inhibition test, this formulation caused a 88 millimeter Zone of Inhibition for Staphylococcus aureus and a 88 millimeter Zone of Inhibition for Escherichia coli and a 150 millimeter Zone of Inhibition for Bacillus anthracis.

In the Plate Cidality test there was no growth observed for any of these organisms.

This formulation was tested in a time to kill study with about a 7% spore population bacteria. Approximately 1.0 ml of Formulation 14 were used in the study into which 10/8 cfu/ml was added and viable counts were measured over time intervals. The time intervals were 1, 5, 15, 30 minutes and 1, 2, 4, 6, 8, 12, 24 hours. Time intervals might be slightly different for different runs. At the designated time intervals about 1/1000 of an ml of solution was subcultured onto Petri dishes coptaining TSAR (triplicate soy agar) with 5% Sheep Blood. The plates were incubated for about 24 hours and the number of colonies were counted if the counts were less than 300, otherwise counts were recorded as TNTC (too numerous to count). The results were recorded.

The results were as follows:

| Time | | Remaining Colonies |
|---|---|---|
| 1 | min | 0 |
| 5 | min | 0 |
| 10 | min | 0 |
| 30 | min | 0 |
| 1 | hour | 0 |
| 2 | hours | 0 |
| 3 | hours | 0 |
| 4 | hours | 0 |
| 6 | hours | 0 |
| 12 | hours | 0 |
| 24 | hours | 0 |

Blank control Heavy growth, too numerous to count

### EXAMPLE 57

An all peroxy formulation (Formulation 57) with no Triclosan which is stable and efficacious contained the following components:

| Component | Mass (grams) |
|---|---|
| Hydrogen peroxide 3% | 3.15 |
| stabilized aqueous solution | |
| cocobetaine - low salt | 10.70 |
| version | |
| Lauryl amine oxide | 0.34 |
| citric acid | 0.10 |
| t-butyl hydroperoxide 70% | 3.22 |
| Peracetic acid - 30% | 7.50 |

The total mass of the above formulation is 25.00 grams and has a pH of about 3.0 and a % solids of about 12%.

The above formulation is diluted at room temperature as follows before use:
1 part of the above formulation with 2 parts of 3% aqueous hydrogen peroxide stabilized

The above formulation and the dilution all were stable and showed no signs of degassing.

Testing of this formulation shows that it has superior antimicrobial efficacy. In the Zone of Inhibition test, this formulation caused a 62 millimeter Zone of Inhibition for Staphylococcus aureus and a 90 millimeter Zone of Inhibition for Escherichia coli and a 90 millimeter Zone of Inhibition for Bacillus anthracis.

In the Plate Cidality test there was no growth observed for any of these organisms.

Formulation 57 was prepared using the following procedure:

A 250 ml beaker equipped with a magnetic stir bar and thermometer is charged with 3.14 grams of 3% stabilized aqueous hydrogen peroxide, 10.70 grams of cocobetaine—low salt version, 0.34 grams of lauryl amine oxide, 0.1 grams of citric acid, The contents of the beaker are stirred and heated to about 60-63° C. The contents of the beaker were held at 60-63° C. for 15 minutes with stirring. The contents of the beaker are cooled to room temperature and some of the surfactant begins to gel or solidify. To the mixture are added at room temperature 3.21 g of 70% t-butyl hydroperoxide and stirred for about 15 minutes to form a clear and homogeneous solution. Then 7.50 grams of 30% peracetic acid are added with stirring at room temperature. The solution is stirred an additional 15 minutes at room temperature. The formulation has a pH of about 3.0. The formulation is stable and has no signs of degassing.

We claim:

1. An anti-microbial composition comprising:
at least one surfactant present in a concentration from about 3% to about 95% by weight;
an acid present in a concentration of up to about 20% by weight;
at least one non-cationic anti-microbial agent and the balance water,
wherein the amount of anti-microbial agent is effective to produce a Zone of Inhibition of from 15 to 150 mm and for the composition to exhibit a Plate Cidality of no growth against each of Staphylococcus aureus ATCC 6538, Escherichia coli ATCC 8739, Pseudomonas aeruginosa ATCC 9027 and Candida albicans ATCC 10231 at aqueous dilutions up to 1:2000 eliminate”) and is CPE negative.

2. The antimicrobial composition of claim 1 wherein the Zone of Inhibition is in the range of from about 30 to 110.

3. The antimicrobial composition of claim 2 wherein the Zone of Inhibition is in the range of from about 45 to 75.

4. The antimicrobial composition of claim 1 which is effective to produce a Zone of Inhibition against Staphylococcus aureus ATCC 6538 of from 50 to 65.

5. The antimicrobial composition of claim 1 which is effective to produce a Zone of Inhibition against Escherichia coli ATCC 8739 of from 50 to 65.

6. The antimicrobial composition of claim 1 which is effective to produce a Zone of Inhibition against Bacillus subtilus and Bacillus anthracis of from 65 to 90 and a Plate Cidality of no growth against Bacillus subtilus and Bacillus anthracis.

7. The anti-microbial Formulation of claim 1, wherein said surfactant is selected from the group consisting of amphoteric surfactant, cationic surfactant, nonionic surfactant, anionic surfactant, and combinations thereof.

8. The anti-microbial composition of claim 1, wherein said compositions includes a first surfactant and a second surfactant.

9. The anti-microbial Formulation of claim 8, wherein said first surfactant is an amphoteric surfactant and said second surfactant is an anionic surfactant.

10. The anti-microbial composition of claim 1, wherein said surfactant is selected from the group consisting of alkylamine oxides, alkyamidopropyl amine oxides, alkyl betaines, alkyamidopropyl betaines, and sultaines.

11. The anti-microbial composition of claim 10, wherein said alkylamine oxide is selected from the group consisting of octyl amine oxide, decyl amine oxide, lauryl amine oxide, iso-dodecyl amine oxide, myristyl amine oxide, cetyl amine oxide, oleamine oxide, stearyl amine oxide, and palmitamine oxide.

12. The anti-microbial composition of claim 10, wherein said alkylamidopropyl amine oxide is selected from the group consisting of laurylamidopropyl amine oxide, cocamidopropyl amine oxide, stearamidopropyl amine oxide, germamidopropyl amine oxide.

13. The anti-microbial composition of claim 10, wherein said alkyl betaine is selected from the group consisting of octyl betaine, lauryl betaine, cocobetaine, cetyl betaine, oleyl betaine, and tallow dihydroxylethyl glycinate.

14. The anti-microbial composition of claim 10, wherein said alkylamidopropyl betaine is selected from the group consisting of caprylamidopropyl betaine, capramidopropyl betaine, lauamidopropyl betaine, cocamidopropyl betaine, isostearamidopropyl betaine, wheatgermaidopropyl betaine, and coco/sunfloweramidopropyl betaine.

15. The anti-microbial composition of claim 10, wherein said sultaine is selected from the group consisting of cocamidopropyl hydroxysultaine and lauryl hydroxysultaine.

16. The anti-microbial composition of claim 1, wherein said surfactant is selected from the group consisting of alkali metal salts of alkylamphoacetates, dialkali metal salts of alkylamphoacetates, alkali metal salts of alkylamphopropionates, dialkali metal salts of alkylamphopropionates, alkanolamides, disodium alkyl sulfosuccinates, monsodium diakyl sulfosuccinates, disodium alkylethoxy sulfosuccinates, sodium ethoxydimethicone sulfosuccinates, disodium propoxydimethicone sulfosuccinates, disodium alkylamido MEA sulfosuccinates, disodium alkylamido MPA sulfosuccinates, sodium alkylamidopropoxy sulfosuccinates, alkylamidopropylmorpholine lactate, and ethylene oxie/propylene oxide diblock and triblock surfactants.

17. The anti-microbial composition of claim 1, wherein said acid is an organic acid or an inorganic acid.

18. The anti-microbial composition of claim 17, wherein said organic acid is citric acid.

19. The anti-microbial composition of claim 17, wherein said inorganic acid is phosphoric acid.

20. The anti-microbial composition of claim 1, wherein said salt is an organic salt or an inorganic salt.

21. The anti-microbial composition of claim 20, wherein said organic salt is a citrate.

22. The anti-microbial composition of claim 20, wherein said inorganic salt is ammonium bifluoride.

23. The anti-microbial composition of claim 1, wherein said non-cationic antimicrobial agent is selected from the group consisting of phenolics, halogenated phenolics, hogenated diphenyl ethers, halogenated carbonilides, water soluble or water insoluble peroxy oxidizing agents, and combinations thereof.

24. The anti-microbial composition of claim 23, wherein said peroxy oxidizing agent is selected from the group consisting of peroxides, peresters, peracids, and persulfates.

25. The anti-microbial composition of claim 24, wherein said peroxy oxidizing agent is the group consisting of hydrogen peroxide, t-butyl hydroperoxide, cumene hydroperoxide and peracetic acid.

26. The anti-microbial composition of claim 1, wherein said non-cationic anti-microbial agent is Triclosan.

27. The anti-microbial composition of claim 7, wherein said amphoteric surfactant is cocobetaine.

28. The anti-microbial composition of claim 7, wherein said surfactant is an alkyl amine oxide.

29. The anti-microbial composition of claim 28, wherein said surfactant is lauryl amine oxide.

30. The antimicrobial composition of claim 7 wherein said anionic surfactant is a sulfosuccinate.

31. The anti-microbial composition of claim 1, wherein said anti-microbial composition possesses broad spectrum anti-microbial activity.

32. The anti-microbial composition of claim 31, wherein said broad spectrum anti-microbial activity includes anti-bacterial activity against Gram-negative bacteria and Gram-positive bacteria.

33. The anti-microbial composition of claim 1, wherein said anti-microbial composition is a clear and homogeneous composition.

34. A method of formulating an anti-microbial composition, comprising the steps of:
forming an admixture of water, and at least one surfactant;
heating said admixture to the melting point of a first solid non-cationic anti-microbial agent;
adding the non-cationic anti-microbial agent to said admixture while rapidly stirring at this melting point temperature; and
cooling said admixture to approximately room temperature,

35. The method of claim 34 wherein at least one acid is added to the admixture of water and surfactant.

36. The method of claim 34 wherein a second noncationic antimicrobial is added at room temperature after said cooling step.

37. The method of claim 34, further comprising the step of adding a peroxy oxidizing agent to said admixture after said cooling step.

38. A method of inhibiting the growth of microbes on a surface comprising the step of applying the composition of claim 1 to a surface.

39. A method of inhibiting the growth of microbes in a liquid, comprising the step of mixing the composition of claim 1 into a liquid in which microbial growth is desired to be inhibited.

40. A method of inhibiting the growth of microbes on the skin comprising applying the composition of claim 1 in combination with water to the skin.

41. A method of formulating an anti-microbial composition composed of:
at least one surfactant present in a concentration from about 5% to about 95% by weight;
at least one non-cationic anti-microbial agent present in a concentration of up to about 20% by weight; and
the balance water; comprising the steps of:
forming an admixture of said water, and said at least one surfactant;
heating said admixture to the melting point of a first non-cationic anti-microbial agent;
adding said non-cationic anti-microbial agent to said admixture while rapidly stirring at this melting point temperatures; and
cooling said admixture to approximately room temperature.

42. The method of claim 41 which further comprises adding at least one acid to the admixture in a concentration of up to about 20% by weight.

43. A method of formulating an anti-microbial composition composed of:
at least one surfactant present in a concentration from about 5% to about 95% by weight;
at least one non-cationic anti-microbial agent present in a concentration of up to about 20% by weight; and
the balance water; comprising the steps of:
mixing a solid antimicrobial agent with a liquid antimicrobial agent capable of dissolving or liquefying the solid antimicrobial agent, at ambient temperatures and agitating the mixture until a clear homogeneous Formulation is produced.

44. The method of claim 43 which further comprises adding at least one acid to the admixture in a concentration of up to about 20% by weight.

45. The method of claim 44 in which an aqueous solution of a peroxide is the liquid antimicrobial agent.

46. An antimicrobial composition obtained by the method of claim 43.

47. A method for killing microbes on a surface comprising applying antimicrobial cidal effective amount of the composition of claim 1 to the surface.

48. A method for rendering the atmosphere of an enclosed space antimicrobial comprising applying an antimicrobial effective amount of the composition of claim 1 to the atmosphere in the enclosed space.

49. An antimicrobial composition obtained by the method of claim 45.

50. An antimicrobial formulation comprising about 6.67 wt. % cocobetaine—low salt version, about 0.13 wt. % lauryl amine oxide, about 0.13 wt. % citric acid, about 0.73 wt, % triclosan, about 3.00 wt. % t-butyl hydroperoxide and about 13 ppm iron sulfate, the balance water.

51. An antimicrobial formulation comprising about 4% cocobetaine-low salt version, about 0.13% lauryl amine oxide, about 0.73% Triclosan, about 3% t-butyl hydroperoxide, about 2% peracetic acid and about 2% stabilized hydrogen peroxide, the balance water.

52. A method for preparing a stable peroxy antimicrobial formulation comprising adding from about 5 to 70% by weight of stabilized hydrogen peroxide solution to a surfactant solution, heating the mixture at approximately 60° C. for about 8 to 12 minutes, adding a noncationic antimicrobial agent with rapid stirring to obtain a clear homogenous low viscosity solution, stirring the solution at about 60-63° C. for 10 minutes, cooling the formulation to room temperature, adding an anti-microbial peroxide component and stirring the mixture for one to five minutes to form a clear and homogeneous solution which does not degas.

53. The method of claim 52 wherein the amount of stabilized hydrogen peroxide added is between 15 and 50% by weight of stabilized hydrogen peroxide.

54. The method of claim 52 wherein the resulting formulation is diluted with a solution of stabilized hydrogen peroxide solution, an aqueous solution of iron sulfate or water.
